(19) United States
(12) Patent Application Publication
(10) Pub. No.: US 2011/0137061 A1
(43) Pub. Date: Jun. 9, 2011

(54) NANOINK FOR FORMING ABSORBER LAYER OF THIN FILM SOLAR CELL AND METHOD OF PRODUCING THE SAME

(76) Inventor: Chi-Jie Wang, Tainan City (TW)

(21) Appl. No.: 12/634,301

(22) Filed: Dec. 9, 2009

(51) Int. Cl. C07C 217/00; C07F 1/08; C07F 5/06

(52) U.S. Cl. 556/110; 556/170; 556/1; 564/504

(57) ABSTRACT

A nanoink composition for forming an absorber layer of a thin film solar cell comprises particles and a volatile chelating agent mixing with the particles. The particles contain one or more elements selected from group IB and/or IIIA and/or VIA. In the present invention, the volatile chelating agent is a polyetheramine which can alternatively be monoamine compounds, diamine compounds and triamine compounds and has a molecular weight of from about 100 to about 4,000. Accordingly, the particles can be reacted mutually into a single composition while the existence of the volatile chelating agent.

### FIELD OF THE INVENTION

The present invention relates to a composition and method for preparing thin films of semiconductors for photovoltaic applications and more particularly a composition and method for preparing Group IB IIIA VIA thin films for thin film solar cells.

### BACKGROUND OF THE INVENTION

Solar cells are sorts of photovoltaic devices converting sunlight to useable electrical power. Because of improvement in conversion efficiency of cells and reduction of costs for manufacturing products in commercial scale, the interest in solar cells, has obviously expended in recent years. The most common material applied into the solar cells is silicon, which is in form of a single or polycrystalline thick wafer. However, although the silicon-based solar cells hold the high conversion efficiency at over 20%, a significant level of thickness to absorb the sunlight has been retained so that the decrease of manufacturing cost and the expanse of application on irregular surface are restricted.

Another type of solar cells, namely the “thin-film”, distinguished from the silicon-based cells has been developing rapidly due to the lower material cost and the competitive conversion efficiency. The typical structure of a thin-film solar cell essentially includes a substrate, a back contact layer, a p-type semiconductor absorber layer, an n-type junction buffer layer, and a transparent layer. Presently, one of most potential absorber layer applied in thin-film solar cells uses a copper indium diselenide (CuInSe2, CIS) compound or the variants copper indium gallium diselenide (Cu(In, Ga)Se2, CIGS) and any of these compounds with sulfur replacing the selenium. CIGS or CIS cells have demonstrated the highest efficiencies and good stability as compared to other absorber layer compounds. Sometimes the acronym CIS and CIGS have been in common use in literature, so CIGS is used here in an expanded meaning to represent the entire group of CIS based alloys.

For producing a CIGS absorber layer, one of the conventional techniques that yielded high-quality CIGS layer for solar cell fabrication was co-evaporation of Cu, In, Ga and Se onto a heated substrate in a vacuum. Another technique is a two-stage process that after formation of Cu, In and Ga films on a substrate by means of sputtering or vapor deposition selenization method under Se or H2Se is reacted with the precursor at elevated temperature. Among them, although the vacuum deposition has an advantage of making a high-efficient absorption layer, it shows low materials utilization when making a large-sized absorption layer and also needs expensive equipment. Besides, hydrogen selenide is the most commonly used selenium bearing gas, which is extremely toxic to humans and requires great care in its use.

On account of the disadvantages of the vacuum deposition, methods for formation of CIGS layers using printing processes to coat an ink containing a metal oxide mixture particles on a substrate at a high temperature are now proposed, which allows one to make a large-sized absorption layer uniformly and reduces production costs in manufacturing solar cells, but because the metal oxide precursor is very stable chemically and thermally to form large crystals the low efficiency of the absorption layer would be shown.

### SUMMARY OF THE INVENTION

Accordingly, it is a primary object of the present invention to solve the aforesaid disadvantages by providing a nanoink composition to simplify the process of forming a CIGS/CIS thin film for solar cells by only a printing process.

To achieve the foregoing object, the present invention provides a nanoink composition for forming an absorber layer of a thin film solar cell comprising particles and a volatile chelating agent mixing with the particles. The particles contain one or more elements selected from group IB and/or IIIA and/or VIA. The volatile chelating agent is a polyetheramine which can be alternatively chosen from monoamine compounds, diamine compounds, and triamine compounds.

Furthermore, the present invention provides a method for producing the aforesaid nanoink composition for forming an absorber layer of a thin film solar cell. The method comprises the steps of:
a) obtaining powders containing a particle mixture of at least one elements or the salts from group IB and/or IIIA and/or VIA;
b) adding a volatile chelating agent mixing with the powders, wherein the volatile chelating agent is a polyetheramine which is selected from the group consisting of monoamine compounds, diamine compounds, and triamine compounds and has a molecular weight of from about 100 to about 4000; and
c) heating the volatile chelating agent to a reflux temperature at which the volatile chelating agent is boiling, and reacting the boiling volatile chelating agent with the powders in an inert gas environment.

According to the nanoink composition for forming an absorber layer of a thin film solar cell and the method of producing the same, CIGS/CIS thin film can be formed only by a simple coating and printing process without requirement of alternative vacuum processing or complex equipment. Particularly, the method is applied in a single-stage process instead of the conventional multiple-stage process so that reduction of manufacturing costs is capable of accomplishment. In addition, the volatile chelating agent is easily removed without any residues in an absorber layer and provides certain viscosity to act as a binder during coating or printing process, and thus any viscosity increaser would not be added further, even in a coating or printing processes afterward.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 shows the X-ray diffraction (XRD) pattern of the powder product in Example 1 of the present invention.

FIG. 2 shows the X-ray diffraction (XRD) pattern of the powder product in Example 2 of the present invention.

FIG. 3 shows the TEM image of the powder product in Example 1 of the present invention.

FIG. 4 shows the TEM image of the powder product in Example 2 of the present invention.

FIG. 5 shows the SEM image of the dense film in Example 1 of the present invention.

FIG. 6 shows the SEM image of the dense film in Example 2 of the present invention.

### DETAILED DESCRIPTION OF THE PREFERRED EMBODIMENTS

The present invention discloses a nanoink composition for forming an absorber layer of a thin film solar cell comprising particles and a volatile chelating agent mixing with the particles. The particles contain one or more elements selected from group IB and/or IIIA and/or VIA. The group IB elements include copper, silver and gold. The group IIIA elements include aluminum, gallium, indium and thallium. And the VIB group element is selenium or sulfur. In the present invention, the particles can also alternatively include the salts of groups IB, IIIA and VIA, such as CuCl, InCl3, GaCl3, CuBr, InBr3, GaBr3, CuI, InI3 and GaI3.

The volatile chelating agent of the present invention is a polyetheramine which is selected from the group consisting of monoamine compounds, diamine compounds and triamine compounds. The volatile chelating agent is easily removed by evaporation and avoids the contamination of the absorber layer from any residues. Besides, the volatile chelating agent could react with the particles to form chelation and provide certain viscosity to act as a binder, and thus any viscosity increaser would not be added further, even in a coating or printing processes afterward.

In the present invention, the volatile chelating agent could be chosen from numerous polyetheramine compounds. For example, the monoamine compounds include polyalkylene glycol amines, bis(methyl triethylene glycol)amine, butyl triethylene glycol amine, lauryl polypropylene glycol amine, methyl tripropylene glycol amine, phenol polypropylene glycol amine, polypropylene glycol amine, bis(methyl tripropylene glycol)amine, N-methyl methyl propylene glycol amine, methyl polypropylene glycol amine, bis(methyl polypropylene glycol)amine, tris(methyl diglycol)amine, methyl polyalkylene glycol amine with random or blockwise distribution of the ethylene glycol, and propylene glycol units. The diamine compounds include triethylene glycol diamine, tripropylene glycol diamine, polyethylene glycol diamine, polypropylene glycol diamine, polyalkylene glycol diamine with random or blockwise distribution of ethylene glycol and propylene glycol units, butanediol polyalkylene glycol diamine, and resorcinol polyalkylene glycol diamine. And the triamine compounds include glycerol polyalkylene glycol triamine with random or blockwise distribution of the Ethylene glycol and propylene glycol unit, bis(triethylene glycol amine)amine, and bis(polyalkylene glycol amine)amines.

The present invention further provides a method for producing the nanoink composition forming an absorber layer of a thin film solar cell. The method includes the steps as follow:
a) obtaining particles containing one or more elements selected from group IB and/or IIIA and/or VIA;
b) adding a volatile chelating agent mixing with the particles, the volatile chelating agent is a polyetheramine which is selected from the group consisting of monoamine compounds, diamine compounds and triamine compounds and has a molecular weight of from about 100 to about 4,000; and
c) heating the volatile chelating agent to a reflux temperature at which the volatile chelating agent is boiling, and reacting the boiling volatile chelating agent with the particles in an inert gas environment.

The volatile chelating agent is a polyetheramine as well, which is capable of being chosen from monoamine compounds, diamine compounds, and triamine compounds as the previous examples of the above nanoink composition. The reflux temperature is at range from 180° C. to 300° C., more preferably from 200° C. to 280° C., and the most preferably from 230° C. to 260° C. The boiling volatile chelating agent and the particles react in the inert gas environment for a period of time ranging from 10 hours to 100 hours, more preferably from 15 hours to 60 hours, and the most preferably from 20 hours to 50 hours. The inert gas can be nitrogen, helium or neon.

Certain embodiments of producing process of the present invention will now be described using the following two examples, which are not meant to limit the scope of the invention.

### Example 1

A 500 ml glass reactor was prepared with a magnetic stirrer under the atmosphere of N2 for 30 minutes. A mixture of 15 g copper metal powders, 19.2 g indium powders, 4.8 g gallium and 40.2 g selenium powders (all the elements are in 99.99% purity) was added into the glass reactor, wherein the gallium was preheated at the temperature of 40-50° C. for 20-30 minutes to the melting state before dropping into the reactor. Then a polyetheramine (JEFFAMINE®D-230 Polyetheramine, HUNTSMAN) 300 g, with difunctional, primary amine with an average molecular weight of about 230, was added into the reactor as well. The polyetheramine and the mixture were mixed completely for 2-3 hours to remove the inherent oxygen and water vapor. The mixed polyetheramine and the elements, including copper, indium, gallium and selenium, were heated to reflux at the temperature about 230-250° C. for 40 hours. Then the reactor was cooled down to the room temperature and black liquid products of CIGS were generated.

10 g of all the collected black liquid products were stirred with 200 g ethanol for 2 hours and then transferred into a filter paper (ADVANTEC No. 5A). The leach was washed with 200 ml pure water and 200 ml ethanol and then dried at 40° C. under a vacuum about 0.1 torr for 10 hours to obtain the final product, 2 g CIGS black powder.

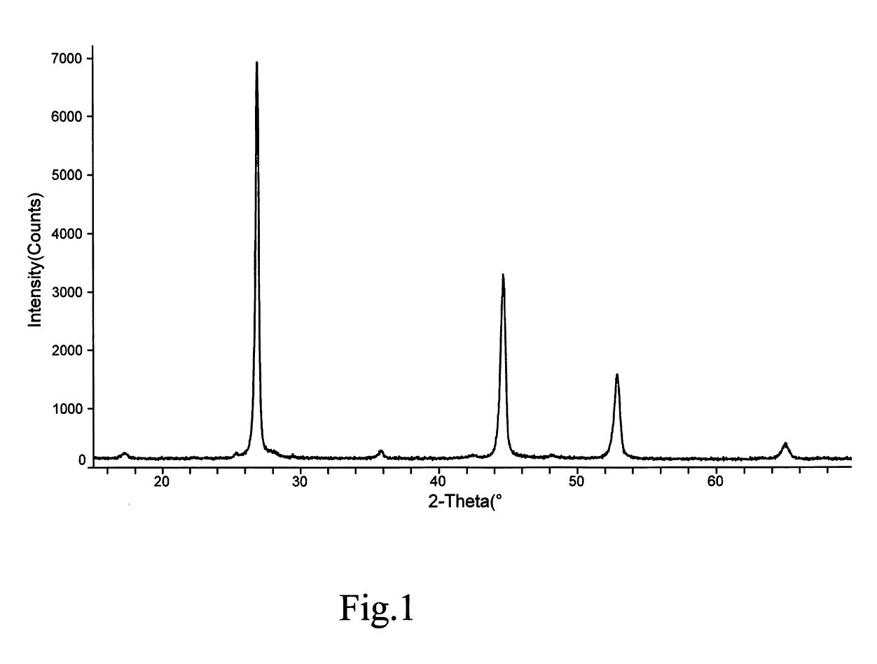

The CIGS black powder formed as described in Example 1 was identified through an X-ray diffraction (XRD) experiment. The XRD pattern was obtained by using a Rigaku 18 kW Rotating Anode X-ray Generator and shown in FIG. 1. Referring to FIG. 1, all the peaks in the XRD pattern could be indexed to a tetragonal chalcopyrite structure, strongest diffraction peak around 2 theta at about 26.7 degrees corresponds to diffraction from 112 plane, while the other peaks at 2 theta at about 44.35 and 52.7 degrees corresponds to diffraction from (220), (204) and (312) planes. These planes are referred to a typical CIS/CIGS crystalline lattice.

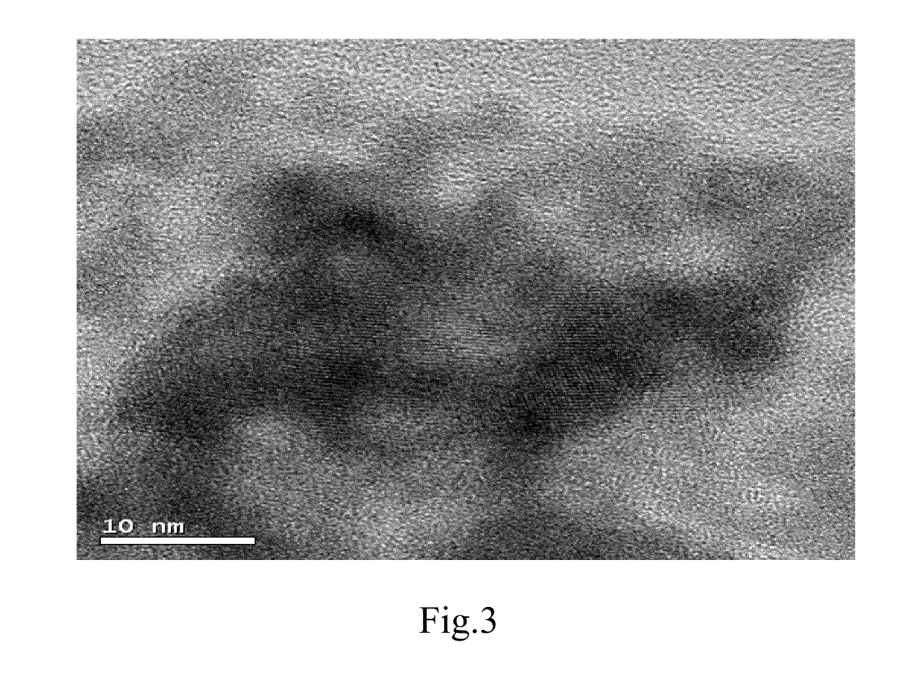

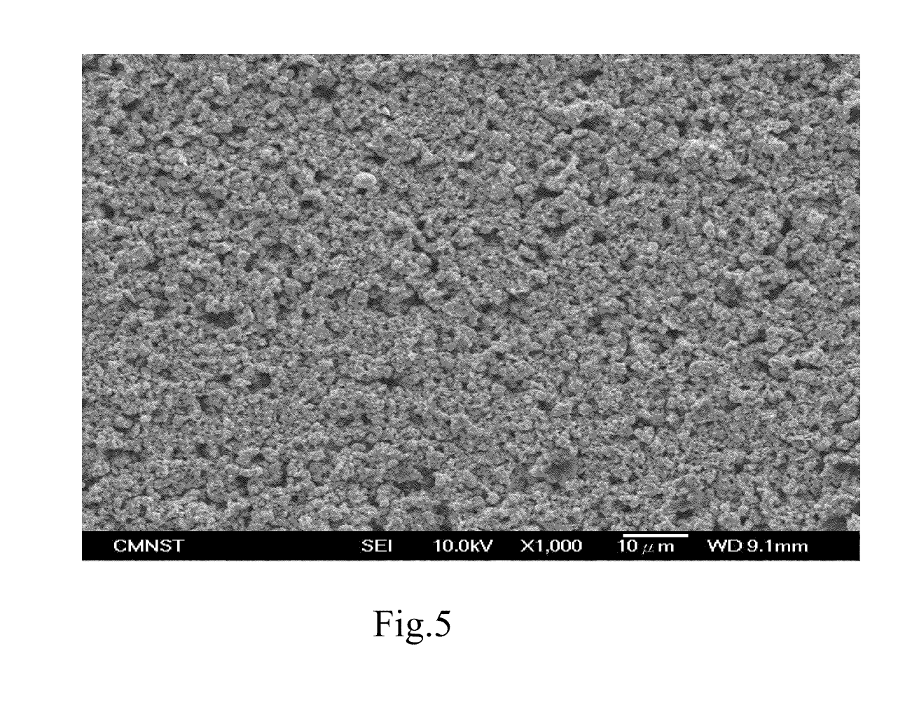

In addition, the average particle size of the black powder and the grain size distribution of the dense film of the black liquid products could be determined separately by a transmission electron microscopy (TEM) analysis, which was performed by using a JEOL JEM-1400 120 kv TEM, and a scanning electron microscopy (SEM) analysis, which was performed by using a Hitachi S4100 FE-SEM, and the results are respectively shown in FIGS. 3 and 5. FIG. 3 illustrates that the average particle size of the black powder generated is about smaller than 10 nm. And FIG. 5 shows the grain size of the dense film is 1-2 m.

### Example 2

The second embodiment chooses another polyetheramine as the volatile chelating agent to produce a nanoink composition.

A 500 ml glass reactor is prepared with a magnetic stirrer under the atmosphere of N2 for 30 minutes. 15 g copper metal powders, 19.2 g indium powders, 4.8 g gallium and 40.2 g selenium powders (all the elements are in 99.99% purity) are added into the glass reactor, wherein the gallium were preheated at the temperature of 40-50° C. for 20-30 minutes to the melting state before dropping into the reactor. Then add another polyetheramine (JEFFAMINE®D-400 Polyetheramine, HUNTSMAN) 300 g with difunctional, primary amine with an average molecular weight of about 430. The polyetheramine and the mixture of the elements are mixed completely for 2-3 hours to remove the inherent oxygen and water vapor. The mixed polyetheramine and the elements, including copper, indium, gallium and selenium, are heated to reflux at the temperature about 240-260° C. for 40 hours. Then the reactor is cooled down to the room temperature and black liquid products of CIGS are generated.

10 g of all the collected black liquid products are stirred with 200 g ethanol for 2 hours and then transferred into a filter paper (ADVANTEC No. 5A). The leach is washed with 200 ml pure water and 200 ml ethanol and then dried at 40° C. under a vacuum about 0.1 torr for 10 hours to obtain the final product, 1.9 g black CIGS powder.

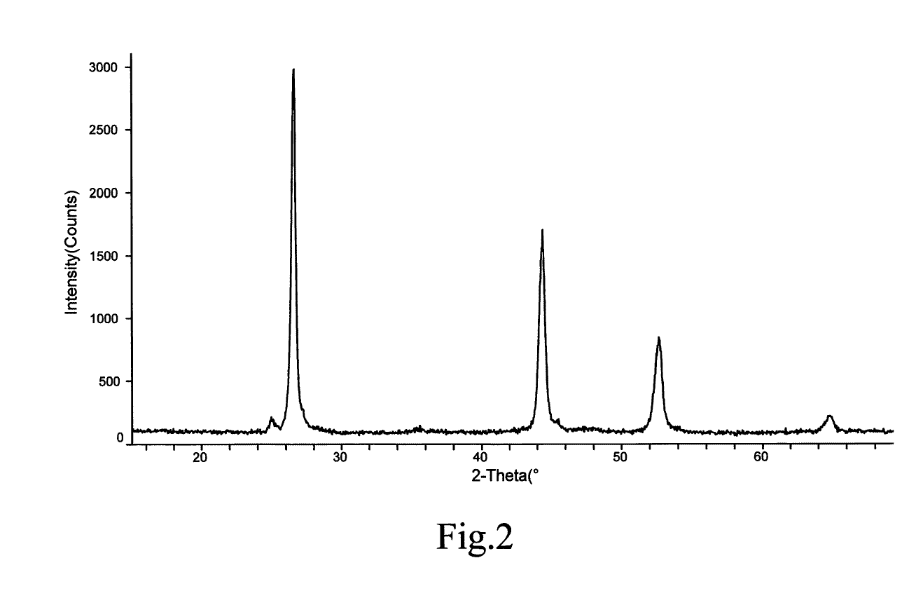

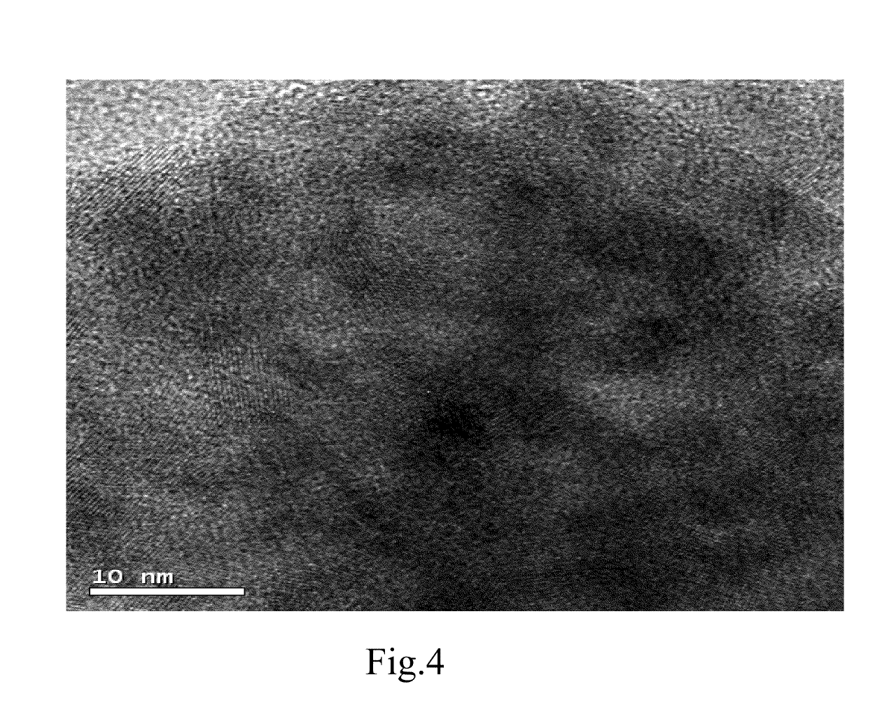

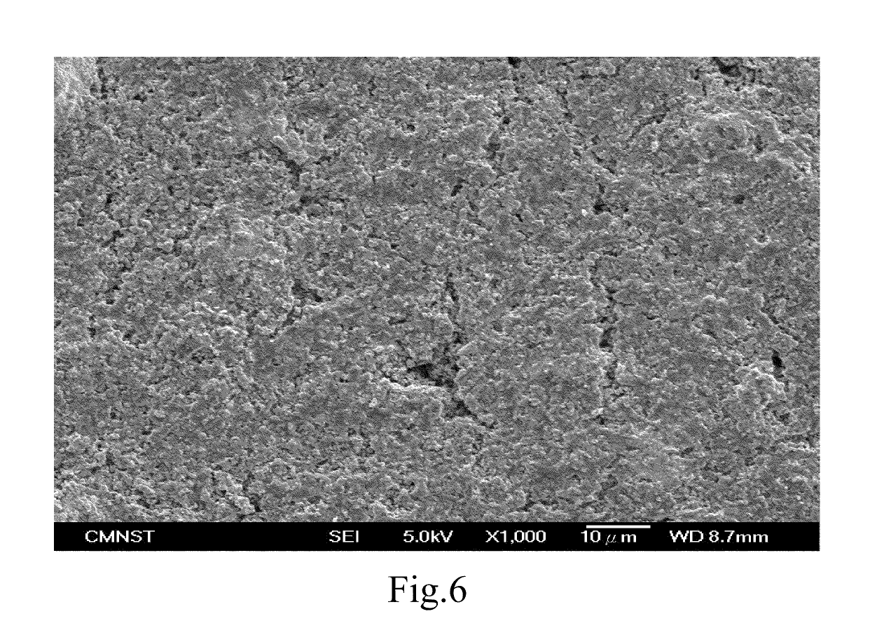

The black powder formed as described in Example 2 was also identified through an X-ray diffraction (XRD) experiment as previous description and the XRD pattern was shown in FIG. 2. Referring to FIG. 2, the strongest peaks around 2 theta in the XRD pattern have similar diffraction to Example 1 and are referred to the typical CIS/CIGS crystalline lattice. As for the average particle size of the black powder and the grain size distribution of the dense film of the black liquid products in Example 2, the results are also determined by the transmission electron microscopy (TEM) analysis and the scanning electron microscopy (SEM) analysis and respectively shown in FIGS. 4 and 6. FIG. 4 illustrates that the average particle size of the black powder generated is about smaller than 10 nm. And FIG. 6 shows the grain size of the dense film is 2-5 m.

As apparent from the foregoing, the method of the present invention is capable of producing a nanoink composition for forming a CIGS film. The volatile chelating agent is a polyetheramine, such as monoamine compound, diamine compound and triamine compound, which easily dissolves the reactant elements or salts, such as selenium and copper, so that the CIGS film can be produced in a simple single-stage process instead of the conventional multiple-stage process which requires alternative vacuum processing or complex equipment.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A nanoink composition for forming an absorber layer of a thin film solar cell, comprising:
particles containing one or more elements selected from group IB and/or IIIA and/or VIA; and
a volatile chelating agent mixing with the particles, wherein the volatile chelating agent is a polyetheramine which is selected from the group consisting of monoamine compounds, diamine compounds and triamine compounds and has a molecular weight of from about 100 to about 4,000.

2. The nanoink composition of claim 1, wherein the group IB element is copper.

3. The nanoink composition of claim 1, wherein the group IIIA element is aluminum, gallium, or indium.

4. The nanoink composition of claim 1, wherein the group VIA element is selenium or sulfur.

5. The nanoink composition of claim 1, wherein the monoamine compounds are selected from the group of alkyl polyalkylene glycol amines, bis(methyl triethylene glycol)amine, butyl triethylene glycol amine, lauryl polypropylene glycol amine, methyl tripropylene glycol amine, phenol polypropylene glycol amine, polypropylene glycol amine, bis(methyl tripropylene glycol)amine, N-methyl methyl propylene glycol amine, methyl polypropylene glycol amine, bis(methyl polypropylene glycol)amine, tris(methyl diglycol)amine, methyl polyalkylene glycol amine with random or blockwise distribution of the ethylene glycol, and propylene glycol units.

6. The nanoink composition of claim 1, wherein the diamine compounds are selected from the group of triethylene glycol diamine, tripropylene glycol diamine, polyethylene glycol diamine, polypropylene glycol diamine, polyalkylene glycol diamine with random or blockwise distribution of ethylene glycol and propylene glycol units, butanediol polyalkylene glycol diamine, and resorcinol polyalkylene glycol diamine.

7. The nanoink composition of claim 1, wherein the triamine compounds are selected from the group of glycerol polyalkylene glycol triamine with random or blockwise distribution of the Ethylene glycol and propylene glycol unit, bis(triethylene glycol amine)amine, and bis(polyalkylene glycol amine)amines.

8. A method for producing a nanoink composition for forming an absorber layer of a thin film solar cell, comprising the steps of:
a) obtaining particles containing one or more elements selected from group IB and/or IIIA and/or VIA;
b) adding a volatile chelating agent mixing with the particles, wherein the volatile chelating agent is a polyetheramine which is selected from the group consisting of monoamine compounds, diamine compounds and triamine compounds and has a molecular weight of from about 100 to about 4,000; and
c) heating the volatile chelating agent to a reflux temperature at which the volatile chelating agent is boiling, and reacting the boiling volatile chelating agent with the particles in an inert gas environment.

9. The method of claim 8, wherein the group IB element is copper.

10. The method of claim 8, wherein the group IIIA element is aluminum, gallium, or indium.

11. The method of claim 8, wherein the group VIA element is selenium or sulfur.

12. The method of claim 8, wherein the monoamine compounds are selected from the group of alkyl polyalkylene glycol amines, bis(methyl triethylene glycol)amine, butyl triethylene glycol amine, lauryl polypropylene glycol amine, methyl tripropylene glycol amine, phenol polypropylene glycol amine, polypropylene glycol amine, bis(methyl tripropylene glycol)amine, N-methyl methyl propylene glycol amine, methyl polypropylene glycol amine, bis(methyl polypropylene glycol)amine, tris(methyl diglycol)amine, methyl polyalkylene glycol amine with random or blockwise distribution of the ethylene glycol, and propylene glycol units.

13. The method of claim 8, wherein the diamine compounds are selected from the group of triethylene glycol diamine, tripropylene glycol diamine, polyethylene glycol diamine, polypropylene glycol diamine, polyalkylene glycol diamine with random or blockwise distribution of ethylene glycol and propylene glycol units, butanediol polyalkylene glycol diamine, and resorcinol polyalkylene glycol diamine.

14. The method of claim 8, wherein the triamine compounds are selected from the group of glycerol polyalkylene glycol triamine with random or blockwise distribution of the Ethylene glycol and propylene glycol unit, bis(triethylene glycol amine)amine, and bis(polyalkylene glycol amine)amines.

15. The method of claim 8, wherein the inert gas is nitrogen, helium or neon.

16. The method of claim 8, wherein the reflux temperature is at the range from 180° C. to 300° C.
